(19) United States
(12) Patent Application Publication
(10) Pub. No.: US 2023/0346737 A1
(43) Pub. Date: Nov. 2, 2023

(54) COMPOUNDS AND METHODS FOR PREVENTION AND TREATMENT OF VIRUS INFECTIONS

(71) Applicant: ARJIL BIOTECH HOLDING COMPANY LIMITED, Hsinchu City (TW)

(72) Inventors: Yeh B WU, Hsinchu City (TW); Jir-Mehng LO, Hsinchu City (TW); Cheng HUANG, Taipei City (TW); Hui-Ju LIANG, Taipei City (TW); Pei-Hsin LIN, Hsinchu County (TW); Hao CHIANG, Taipei City (TW); Wei-Chung CHIOU, Taipei City (TW); Guan-Jhong HUANG, Taoyuan City (TW)

(73) Assignee: ARJIL BIOTECH HOLDING COMPANY LIMITED, Hsinchu City (TW)

(21) Appl. No.: 18/023,415

(22) PCT Filed: Aug. 27, 2021
(86) PCT No.: PCT/US2021/047935

§ 371 Date: Feb. 27, 2023

Related U.S. Application Data
(60) Provisional application No. 63/071,564, filed on Aug. 28, 2020.

(51) Int. Cl. A61K 31/352; A61K 31/351; A61K 31/575; A61K 31/12; A61K 31/7048; A61K 45/06; A61P 31/20

(52) U.S. Cl. CPC A61K 31/352; A61K 31/351; A61K 31/575; A61K 31/12; A61K 31/7048; A61K 45/06; A61P 31/20

(57) ABSTRACT

The present invention pertains to anti viral compounds. The disclosure includes a method for preventing and/or treating a virus infection through the inhibition of a cysteine protease in a virus and/or a sodium taurocholate cotransporting polypeptide in a cell, particularly SARS-COV-2 and hepatitis B virus (HBV). Also provided includes the composition/pharmaceutical composition for preventing and/or treating a virus infection comprising any of the compounds, pharmaceutically acceptable salt thereof, or its mixture, and the use of the compounds.

### CROSS REFERENCE TO RELATED APPLICATION

This application claims benefit of priority from U.S. Provisional Application Ser. No. 63/071,564, filed on Aug. 28, 2020.

### FIELD OF THE INVENTION

The present invention provides some anti-viral compounds and the method and combination/composition-pharmaceutical composition for prevention and treatment of virus infections, particularly the disease caused by coronavirus or hepatitis virus.

### BACKGROUND OF THE INVENTION

Viruses, made of genetic material inside of a protein coating, invade living, normal cells and use those cells to multiply and produce other viruses like themselves, that may cause familiar infectious disease such as flu and warts, or may cause severe illness such as smallpox and acquired immune deficiency syndrome (AIDS).

For example, there are 5 different types of hepatitis viruses i.e., A, B, C, D and E along with X and G. Hepatitis A and E viruses are induced by consumption of pestiferous water and food. However, hepatitis B, C, and D viruses are caused by parental, adjoin with infected bodily fluids. In addition, hepatitis C and D virus infections are also on the increase and effective treatments are needed.

Hepatitis B virus (HBV) causes acute and chronic viral hepatitis in humans. HBV infection is often associated with severe liver diseases, including cirrhosis and hepatocellular carcinoma MCC) [1]. The prevalence of HBV infection in the world is very high. About 350 million individuals are chronically infected, despite the availability of an effective vaccine for more than 25 years. Approximately an 100-fold increase in the relative risk of HCC among HBV carriers compared to non-carriers [2].

An increasing number of patients with HBV infection cannot use the currently approved anti-HBV drugs, including interferon alpha or nucleos(t)ide analogues that inhibit the viral reverse transcriptase, due to the adverse effects and the emergence of drug resistance [3].

Therefore, the search for effective and safe as well as affordable anti-HEW agents aiming at the interference with other steps in the viral life cycle is required to improve the treatment outcome.

HBV is a small DNA virus consisting of a nucleocapsid which protects the 3.2 kb viral genome [4]. The HBV nucleocapsid is surrounded by an envelope, consists of hepatitis B surface antigens (HBsAgs). HBsAgs are encoded in one open reading frame with three in-phase start codons. The MHBsAg has a 55-amino-acid (aa) extension from the S domain that is known as the pre-S2 domain. The LHBsAg has a further 108-aa region that extends from the pre-S2 domain to compose the pit-S1 domain. Recently, sodium taurocholate cotransporting polypeptide (NTCP) was identified as an HBV receptor [5, 6]. Entry of HBV into uninfected hepatocytes has long been proposed as a potential target for antiviral intervention [7]. On the other hand, HepG2.2.1 S cells encompass HBV whole genome, which was widely used to study HBV replication, assembly, and secretion.

The attachment to hepatocyte by HBV during infection has long been proposed to be a potential target for antiviral intervention. It is thought that molecules specifically binding to HBV particles may interfere with viral attachment and hence reduce or block subsequent infection [8].

Insights into the early infection events of human HBV are limited because of the lack of a cell culture system supporting the full replication cycle. To date, two cell types have been shown to be susceptible to HBV infection. One is the human hepatoma cell line HepaRG, which becomes infectable after dimethyl sulfoxide (DMSO)-induced differentiation [7, 9], while the other cell type, normal human primary hepatocytes, is readily infected by HBV [10, 11], but the limited lifetime of the cells in vitro and the lack of a consistent source severely restrict its further application.

Besides, Herpes simplex virus (HSV) also consists of a DNA genome encased within a protein coating. Herpes simplex virus types 1 and 2 (HSV-1 and HSV-2) are the causative agents of human diseases, including gingivostomatitis, pharyngitis, herpes labialis, encephalitis, eye and genital infection [12]. Herpesvirus infections generally involve a mild or asymptomatic primary phase followed by persistence of the virus in a non-replicating latent state or at a clinically undetedable level of replication [13]. Primary infection with HSV-1 most commonly involves the mouth and/or throat resulting in gingivostomatitis and pharyngitis. Following recovery from the primary oropharyngeal infection, the individual retains HSV DNA in the trigeminal ganglion for life and may suffer recurrent attacks of herpes labialis. Studies have also revealed a possible association between some members of the herpesvirus family and periodontal diseases [14]. Human herpesviruses may occur in periodontitis lesions with relatively high prevalence [15]. HSV is related to the severity of periodontal diseases in terms of clinical attachment loss [16]. Viral gingival infections may act to impair host defense mechanisms and thereby set the stage for overgrowth of pathogenic oral bacteria [15, 17].

HSV commonly attacks mucosa, skin, eyes and the nervous system and is capable of infecting a wide variety of cells [18]. Human gingival mucosa organ culture can be infected with HSV-1 and HSV-2 [19]. In addition, human gingival keratinocytes and gingival fibroblasts which are grown in vitro support the multiplication of HSV [20, 21]. HSV-1 encodes viral thymidine kinase, which indirectly metabolizes acyclovir into acyclovir triphosphate, a chain terminator substrate for HSV DNA polymerase and stops viral DNA replication [22]. However, resistance of HSV to acyclovir has been reported in 5-30% of cases [23]). Acyclovir-resistant HSV-1 strains occur frequently in immunocompromised patients, which may result in severe complications [24]. Due to the lack of vaccine, topical microbicides may be an important strategy for preventing HSV transmission.

Severe acute respiratory syndrome (SARS) outbreak in November 1st, 2002 to Jun. 18, 2003 led to 801 deaths in over 29 countries and 8465 probable cases around the world according to the World Health Organization (WHO) [25]. SARS, an enveloped β coronavirus containing positive-sense, single-stranded RNA, has a genome size of about 30 kb, in which open reading frame (ORF) 1a and 1b encode for two respective polyproteins (pps), pp1a and pp1ab [26, 27]. To complete its lifecycle, successful replication and proteolytic processing are imperative [28]. Indeed, the consensus functions of these virus-encoded proteolytic proteins are found in all coronaviruses, specifically papline-like protease (PLpro) and chymotrypsin-like protease (3CLpro) [28]. In proteolytic processing of pp1a and pplab, PLpro and 3CLpro cleave the first three sites and the remaining 11 locations, respectively, yielding a total of 16 nonstructural proteins (nsp1-16) [26, 27]. Thus, 3CLpro inhibition has been regarded as a molecular approach in anti-SARS drug discovery and developments [25, 29].

SARS-COV-2 is a novel coronavirus that spreads rapidly since its identification in patients with severe pneumonia in Wuhan, China (named as COVID-19), has been reported in 25 countries, with nearly 72000 laboratory-confirmed cases and a death toll of 1775 worldwide as of Feb. 17, 2020 [30]. Devastatingly, no drug or vaccine has yet been approved to treat human coronaviruses [31]. Concerning the current outbreak of SARS-CoV-2 and the therapeutic experience of SARS and MERS (another β coronavirus), many studies extensively investigate the possibility of using the existing antiviral agents used for HIV, hepatitis B virus, hepatitis C virus and influenza infections for the treatment or intervention of SARS-COV-2 [31, 32]. In the meantime, SARS-CoV-2 has been characterized as an enveloped, positive-sense, single-stranded RNA β coronavirus, similar to SARS and MERS [31]. Consistent with the characteristics of coronaviruses, SARS-CoV-2 genome encodes structural proteins (e.g., spike glycoproteins), nonstructural proteins (e.g., 3CLpro, PLpro, helicase, RNA-dependent RNA polymerase), and accessory proteins. Regarding the available genomic sequence of SARS-COV-2, SARS and MERS, a high-level conservation of the proteolytic sites and proteolytic enzymes was found, whence repurposing SARS and MERS protease inhibitors for treatment of SARS-COV-2 is worth considering [33]. As 3CLpro plays a pivotal role in SARS, it is reasonable to approach protease inhibition by targeting the 3CLpro of SARS-COV-2 instead of its PLpro to intercept its lifecycle [25, 29, 33].

Currently, disulfiram, an approved drug to treat alcohol dependence, has been reported to inhibit the PLpro of MERS and SARS in cell cultures but has yet been evaluated clinically [31]. In addition, clinical trials of HIV protease inhibitors (lopinavir and ritonavir) in SARS-CoV-2 patients have also commenced, yet it is uncertain if it can effectually inhibit those of SARS-CoV-2, as HIV and β coronavirus proteases belong to the aspartic protease family and the cysteine protease family, respectively [31, 34]. On the other hand, remdesivir (RDV), a nucleotide analog of RNA dependent RNA polymerase inhibitor approved for HIV treatment, is currently under clinical trials in SARS-CoV-2 patients with estimated completion dates in April, 2020; galidesivir, another nucleotide analog of RNA dependent RNA polymerase inhibitor in early-stage clinical studies for HCV treatment, has shown broad-spectrum antiviral activities against severe acute respiratory syndrome (SAKS), Middle East respiratory syndrome (MFRS) in preclinical studies [34, 35]. However, one might expect that a nucleoside analog can elicit toxicity that are still beyond our knowledge [36].

There are yet to find antiviral drugs to prevent or treat human coronavirus infections. There is an urgent need for exploring and developing a safe anti-coronavirus therapy, particularly against SARS-COV-2.

Still, it is desirable to develop a new antiviral therapy or medicament.

### BRIEF SUMMARY OF THE INVENTION

It is unexpectedly found in the present invention that some triterpenes are effective in inhibition of virus infection, especially a Hepatitis B virus (HBV) infection and/or a Herpes simplex virus (HSV) and/or a coronavirus infections, especially SARS-COV-2.

In one object, the present invention provides a method for inhibiting a virus infection comprising administering to a subject in need thereof a pharmaceutical composition comprising a therapeutically effective amount of a compound or pharmaceutically acceptable salt thereof, or its mixture, in which the compound is selected from the group consisting of:

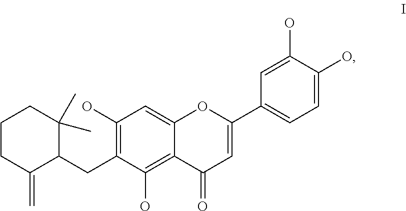

ugonin N having the structure of formula II and its derivatives:

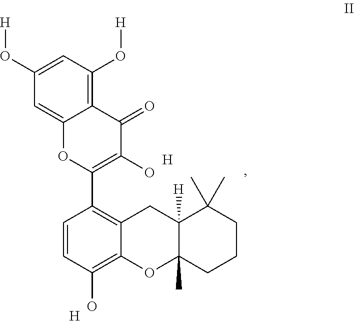

6-(3,4-dihydroxyphenyl)-4-hydroxyhexa-3,5-dien-2-one having the structure of formula III and its derivatives:

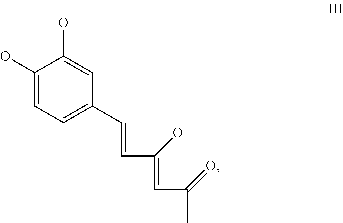 [SMILES: CC(=O)/C=C(O)/C=C/c1ccc(O)c(O)c1]

2-[(E)-2-(3,4-dihydroxyphenyl)ethenyl]-6-hydroxypyran-4-one having the structure of IV and its derivatives:

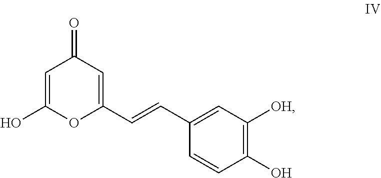 [SMILES: O=c1cc(O)oc(/C=C/c2ccc(O)c(O)c2)c1]

dehydroeburicoic acid having the structure of formula V and its derivatives:

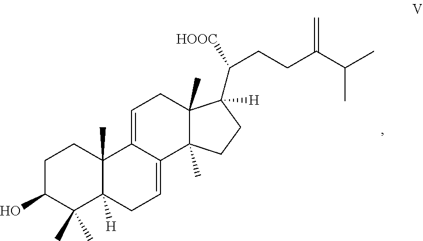

3-O-methyl kaempferol having the structure of formula VI and its derivatives:

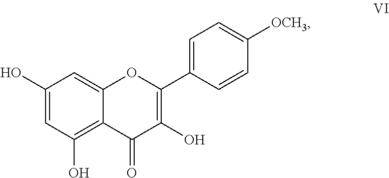 [SMILES: COc1ccc(-c2oc3cc(O)cc(O)c3c(=O)c2O)cc1]

kaempferol-3-O-(3, 4-diacetyl-Alpha-L-rhamnopyranoside having the structure of formula VII and its derivatives:

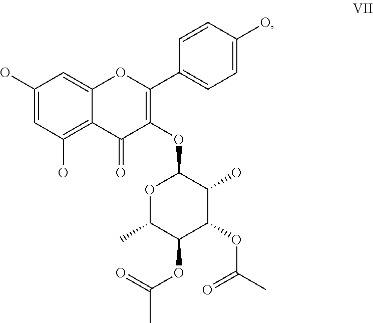

kaempferol-3-O-(2, 4-diacetyl-Alpha-L-rhamnopyranoside having the structure of formula VIII and its derivatives:

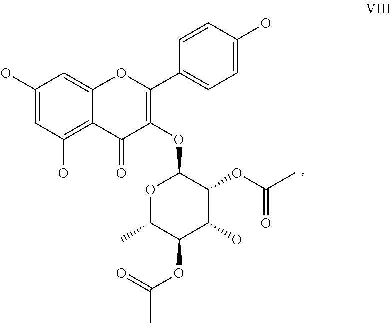 [SMILES: CC(=O)O[C@@H]1[C@@H](O)[C@@H](OC(C)=O)[C@H](Oc2c(-c3ccc(O)cc3)oc3cc(O)cc(O)c3c2=O)O[C@H]1C]

dehydrosulphurenic acid having the structure of formula IX and its derivatives:

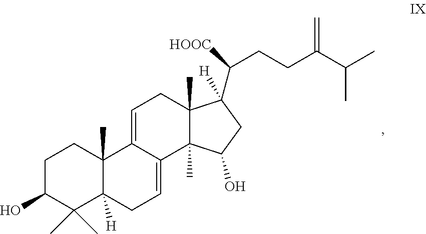 [SMILES: C=C(CC[C@H](C(=O)O)[C@H]1C[C@H](O)[C@@]2(C)C3=CC[C@H]4C(C)(C)[C@@H](O)CC[C@]4(C)C3=CC[C@]12C)C(C)C]

sulphurenic acid having the structure of formula X and its derivatives:

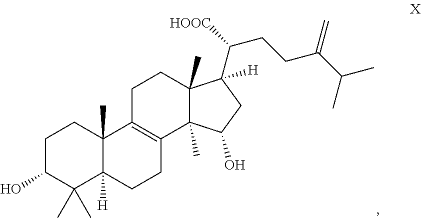 [SMILES: C=C(CC[C@@H](C(=O)O)[C@H]1C[C@H](O)[C@@]2(C)C3=C(CC[C@]12C)[C@@]1(C)CC[C@@H](O)C(C)(C)[C@@H]1CC3)C(C)C]

versisponic acid D having the structure of formula. XI and its derivatives:

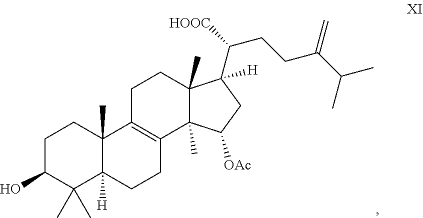 [SMILES: C=C(CC[C@@H](C(=O)O)[C@H]1C[C@H](OC(C)=O)[C@@]2(C)C3=C(CC[C@]12C)[C@@]1(C)CC[C@H](O)C(C)(C)[C@@H]1CC3)C(C)C]

trans-p-menth-6-ene-2,8-diol having the structure of formula XII and its derivatives:

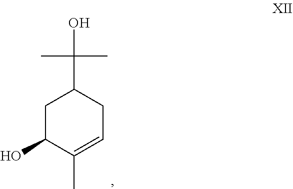

antcin K having the structure of formula XIII and its derivatives:

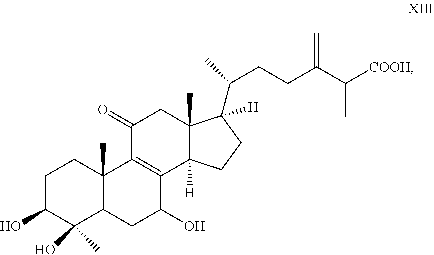 [SMILES: C=C(CC[C@@H](C)[C@H]1CC[C@H]2C3=C(C(=O)C[C@]12C)[C@@]1(C)CC[C@H](O)[C@](C)(O)C1CC3O)C(C)C(=O)O]

and a combination thereof.

In one object, the present invention provides a method for preventing and/or treating a virus infection, comprising administering to a subject in need thereof a compound or a pharmaceutically acceptable salt thereof, or a combination/composition/pharmaceutical composition of two or more of the compounds as set forth above.

In some particular examples of the present invention, the compound is selected from the group consisting of Ugonin J, Ugonin N, (6-(3,4-dihydroxyphenyl)-4-hydroxyhexa-3,5-dien-2-one), (2-[(E)-2-(3,4-dihydroxyphenyl)ethynyl]-6-hydroxypyran-4-one), dehydroeburicoic acid, 3 O-methyl kaempferol, kaempferol-3-O-(3, 4-diacetyl-Alpha-L-Rhamnopyranoside, kaempferol-3-O-(2, 4-diacetyl-Alpha-L-rhamnopyranoside), dehydrosulphurenic acid, sulphurenic acid, versisponic acid D, and trans-p-menth-6-ene-2,8-diol, and antcin K.

In some particular examples of the present invention, the combination is the combination of two or more of the compounds elected from the group consisting of Ugonin J, Ugonin N, (6-(3,4-dihydroxyphenyl)-4-hydroxyhexa-3,5-dien-2-one), (2-[(E)-2-(3,4-dihydroxyphenyl)ethenyl]-6-hydroxypyran-4-one), dehydroeburicoic acid, 3-O-methyl kaempferol, kaempferol-3-O-(3,4-diacetyl-Alpha-L-Rhamnopyranoside, kaempferol-3-O-(2, 4-diacetyl-Alpha-L-rhamnopyranoside), ovatodiolide, dehydrosuiphurenic acid, sulphurenic acid, versisponic acid D, and trans-p-menth-6-ene-2,8-diol, and antcin K.

In one further aspect, the present invention provides a combination/composition/pharmaceutical composition for preventing or treating a virus infection, particularly a coronavirus, e.g., SARS-COV-2, comprising a therapeutically effective amount of any of the compounds set forth in the present invention, or pharmaceutically acceptable thereof, or its mixture, in combination of a pharmaceutically acceptable carrier.

In one further aspect, the present invention provides a composition/pharmaceutical composition for preventing and/or treating a hepatitis virus infection, particularly HBV comprising a therapeutically effective amount of any of the compounds disclosed herein or pharmaceutically acceptable thereof, or its mixture, in combination of a pharmaceutically acceptable carrier.

Optionally, the composition/pharmaceutical composition according to the invention may comprise at least one additional anti-viral therapeutic agent.

In one yet aspect, the present invention provides a use of any of the compounds as set forth in the present invention or pharmaceutically acceptable salts, or its mixture for manufacturing a medicament for preventing or treating a virus infection, particularly coronavirus, e.g., SARS-COV-2.

In one example of the invention, the virus is a hepatitis virus, particularly a hepatitis B virus (HBV).

In one example of the invention, the virus is a herpes simplex virus (HSV).

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the relative 3CLpro activity (%) of AR100-DS1+ARH 101-DS2 (0.25p/0.6FP), and IC50=3.065 μM.

FIG. 2 shows the relative 3CLpro activity (%) of AR100-DS1+ARH 101-DS3 (0.25p/0.6FP), and IC50=2.934 μM.

FIG. 3 shows the IC50 of ARH 020-DS1—SARS-Cov-2=14.93 μM.

FIG. 4 shows the IC50 of ARH 020-DS2—SARS-Cov-2=6.329 μM.

FIG. 5 shows the IC50 of ARH 019-DS1—SARS-Cov-2=19.21 μM.

FIG. 6 shows the IC50 of ARH 019-DS2—SARS-Cov-2=2.487 μM.

FIG. 7 shows the IC50 of ARH 007-DS3—SARS-Cov-2=11.61 μM.

FIG. 8 shows the IC50 of ARH 007-DS4—SARS-Cov-2=18.85 μM.

FIG. 9 shows the IC50 of ARH 007-DS5—SARS-Cov-2=48.22 μM.

FIG. 10 shows the relative 3CLpro activity (%) of AR101-DS2+ARH013-DS1, and IC50=2.409 μM.

FIG. 11 shows the relative 3CLpro activity (%) of ARH007-DS3+ARH013-DS1, and IC50=18.2 μM.

FIG. 12 shows the relative 3CLpro activity (%) of AR100-DS1+ARH007-DS3, and IC50=8.646 μM.

FIG. 13 shows ARH-013-DS1—SARS-Cov-2=32.89 μM.

FIG. 14 shows the effects of AR101-DS2 at 0, 20 and 40 μM on HBsAg secretion of HepG2.2.15 cells (*, P<0.05; **, P<0.01; ***. P<0.001).

FIG. 15 shows the effects of AR101-DS2 at 0, 20 and 40 μM on HBV DNA level in the culture medium of HepG2.2.15 cells (*, P<0.05; **. P<0.01; ***, P<0.001).

FIG. 16 shows the effects of AR101-DS2 at 0, 20 and 40 μM on HBsAg secretion of HuS-E/2 cells (*, P<0.05; **, P<0.01; ***, P<0.001).

FIG. 17 shows the effects of AR101-DS2 at 0, 20 and 40 μM on HBV mRNA expression level of HuS-F/2 cells.

FIG. 18 shows the inhibition effect of AR10l-DS3 at 0, 10, 20 and 100 μM on NTCP (*, P<0.05; **, P<0.01; ***, P<0.001).

FIG. 19 shows the inhibition effect of AR101-DS4 at 0, 10, 20 and 100 μM on NTCP (*, P<0.05; **, P<0.01; ***, P<0.001).

FIG. 20 shows the effects of AR101-DS1+AR101-DS3 at 0, 40 and 80 μM on HBsAg secretion of Hus-E/2 cells (*, P<0.05; **, P<0.01; ***, P<0.001).

FIG. 21 shows the effects of AR101-DS1+AR101-DS3 at 0, 40 and 80 μM on HBV mRNA expression level of Hus-E/2 cells (*, P<0.05; **, P<0.01; ***, P<0.001).

FIG. 22 shows the effects of AR101-DS1+AR101-DS4 at 0, 40 and 80 μM on HBsAg secretion of Hus-E/2 cells (*, P<0.05; **, P<0.01; ***, P<0.00l).

FIG. 23 shows the effects of AR101-DS1+AR101-DS4 at 0, 40 and 80 μM on HBV mRNA expression level of Hus-E/2 cells (*, P<0.05; **, P<0.01; ***, P<0.001).

### DETAILED DESCRIPTION OF THE INVENTION

The above summary of the present invention will be further described with reference to the embodiments of the following examples. However, it should not be understood that the content of the present invention is only limited to the following embodiments, and all the inventions based on the above-mentioned contents of the present invention belong to the scope of the present invention.

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

In the present invention, to evaluate the effect of prospecting drugs on proteolytic processing inhibition in high-throughput, the synthetic peptides labelled fluorescence resonance energy transfer (FRET) pairs were employed as those used in the previous studies, in which the quenched fluorophore is released upon cleavage of the FRET-labelled peptides, generating fluorescent signals that can be monitored in real-time (Chen et al., 2005; Jean et al., 1995; Jo et al., 2020). It is confirmed in the present invention that any or its mixture of the compounds disclosed herein is effective in inhibition of a cysteine protease, particularly 3CLpro of SARS-CoV-2.

The present invention provides a method for preventing and/or treating a virus infection comprising administering to a subject in need thereof a compound or pharmaceutically acceptable salt thereof, or its mixture, in which the compound is one selected from the group consisting of:
ugonin J having the structure of formula I and its derivatives:

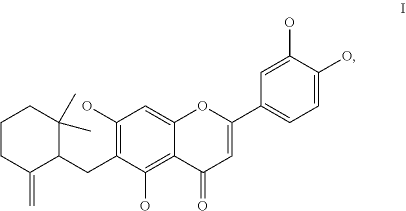

ugonin N having the structure of formula II and its derivatives:

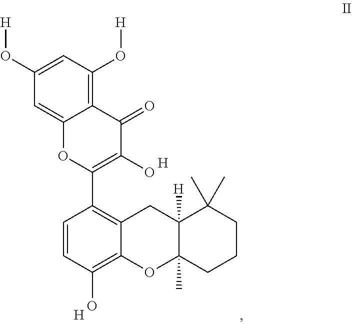

6-(3,4-dihydroxyphenyl)-4-hydroxyhexa-3,5-dien-2-one having the structure of formula III and its derivatives:

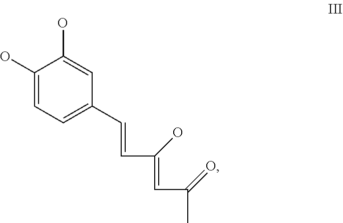 [SMILES: CC(=O)/C=C(O)/C=C/c1ccc(O)c(O)c1]

2-[(E)-2-(3,4-dihydroxyphenyl)ethenyl]-6-hydroxypyran-4-one having the structure of IV and its derivatives:

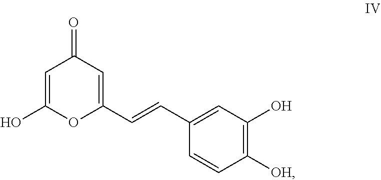 [SMILES: O=c1cc(O)oc(/C=C/c2ccc(O)c(O)c2)c1]

dehydroeburicoic acid having the structure of formula V and its derivatives:

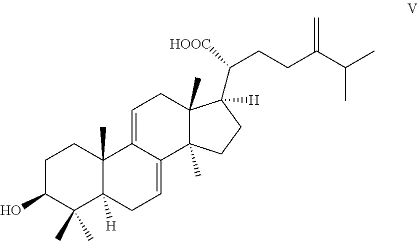

3-O-methyl kaempferol having the structure of formula VI and its derivatives:

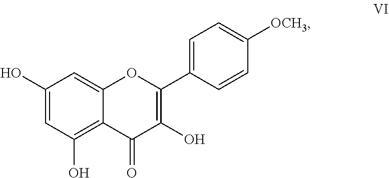 [SMILES: COc1ccc(-c2oc3cc(O)cc(O)c3c(=O)c2O)cc1]

kaempferol-3-O-(3, 4-O-diacetyl-Alpha-L-rhamnopyranoside) having the structure of formula VII and its derivatives:

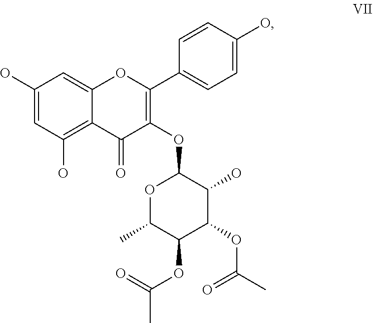

kaempferol-3-O-(2, 4-O-diacetyl-Alpha-L-rhamnopyranoside) having the structure of formula VIII and its derivatives:

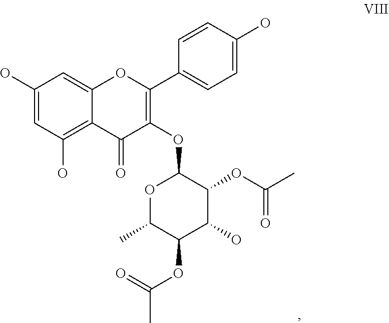 [SMILES: CC(=O)O[C@@H]1[C@@H](O)[C@@H](OC(C)=O)[C@H](Oc2c(-c3ccc(O)cc3)oc3cc(O)cc(O)c3c2=O)O[C@H]1C]

dehydrosulphurenic acid having the structure of formula IX and its derivatives:

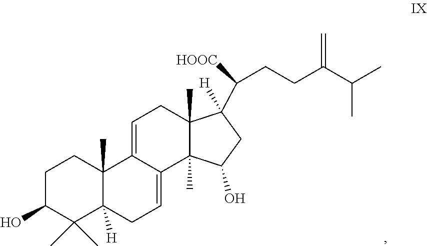 [SMILES: C=C(CC[C@H](C(=O)O)[C@H]1C[C@H](O)[C@@]2(C)C3=CC[C@H]4C(C)(C)[C@@H](O)CC[C@]4(C)C3=CC[C@]12C)C(C)C]

sulphurenic acid having the structure of formula X and its derivatives:

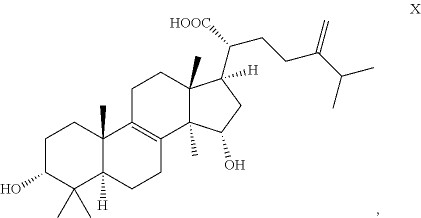 [SMILES: C=C(CC[C@@H](C(=O)O)[C@H]1C[C@H](O)[C@@]2(C)C3=C(CC[C@]12C)[C@@]1(C)CC[C@@H](O)C(C)(C)[C@@H]1CC3)C(C)C]

versisponic acid D having the structure of formula XI and its derivatives:

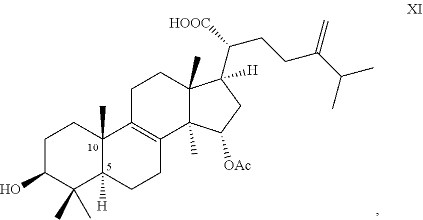

trans-p-menth-6-ene-2,8-diol having the structure of formula XII and its derivatives:

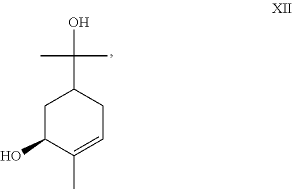

antcin K. having the structure of formula XIII and its derivatives:

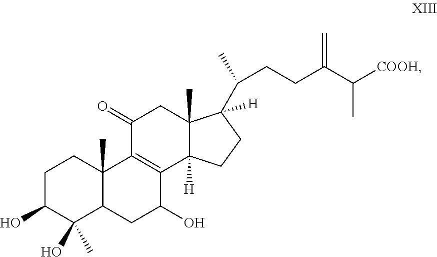 [SMILES: C=C(CC[C@@H](C)[C@H]1CC[C@H]2C3=C(C(=O)C[C@]12C)[C@@]1(C)CC[C@H](O)[C@](C)(O)C1CC3O)C(C)C(=O)O]

and combination thereof.

The present invention also provides a combination/composition/pharmaceutical composition for preventing and/or treating a virus infection, particularly a coronavirus, e;g., SARS-COV-2, which comprises a therapeutically effective amount of a compound as set forth in the present invention and a pharmaceutically acceptable carrier.

The term “virus” as used herein refers to any virus, which is a small infectious agent that replicates only inside the living cells of an organism, which can infect all types of life forms, from animals and plants to microorganisms, including bacterials and archaea. Exemplified viruses include, without limitation, a hepatitis virus, an influenza virus, a herpes simplex virus (HSV), an enterovirus, a rotavirus, a dengue virus, a poxvirus, a human immunodeficiency virus, an adenovirus, a measles virus, a retrovirus, a coronavirus or a norovirus.

The term “Hepatitis virus” as used herein refers to a virus causing hepatitis, particularly a Hepatitis B virus (HBV), Hepatitis C virus (HCV), Hepatitis D virus (HDV).

The term “coronavirus” as used herein refers to a Coronaviruse in the subfamily Orthocoronavirinae, the family Coronaviridae, order Nidovirales, and realm Riboviria, which is enveloped viruses with a positive-sense single-stranded RNA genome and a nucleocapside of helical symmetry. They have characteristic club-shaped spikes that project from their surface, which in electron micrographs create an image reminiscent of the solar corona from which their name derives. Coronaviruses cause diseases in mammals and birds, including humans. In humans, coronaviruses cause respiratory tract infections, including common cold, severe acute respiratory syndrome (SARS), Middle East respiratory syndrome (MERS), and SARS-COV-2.

The term “cysteine protease” as used herein refers to thiol proteases, are enzymes that degrade proteins, sharing a common catalytic mechanism that involves a nucleophilic cysteine thiol in a catalytic triad or duad. One example of cysteine protease in a virus is 3CLpro in SARS-COV-2.

The term “treat,” “treating” or “treatment” as used herein refers to the application or administration of a composition including one or more active agents to a subject afflicted with a disease, a symptom or conditions of the disease, or a progression of the disease, with the purpose to cure, heal, alleviate, relieve, alter, remedy, ameliorate, improve, or affect the disease, the symptoms or conditions of the disease, the disabilities induced by the disease, or the progression of the disease.

The term “prevent,” “prevention” or “preventing” as used herein refers to the prevention of the recurrence, onset, or development of a virus infection, one or more symptoms thereof, or a respiratory condition associated with, potentiated by, or potentiating a coronavirus infection in a subject.

The term “subject” as used herein includes human or non-human animals, such as companion animals (e.g. dogs, cats, etc.), farm animals (e.g. cattle, sheep, pigs, horses, etc.), or experimental animals (e.g. rats, mice, guinea pigs, etc.).

The term “therapeutically effective amount” as used herein refers to an amount of a pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function.

For use in therapy, the therapeutically effective amount of the compound is formulated as a pharmaceutical composition for administration. Accordingly, the invention further provides a pharmaceutical composition comprising a therapeutically effective amount of any or its mixture of these compounds disclosed herein, and one or more pharmaceutically acceptable carriers.

For the purpose of delivery and absorption, a therapeutically effective amount of the active ingredient according to the present invention may be formulated into a pharmaceutical composition in a suitable form with a pharmaceutically acceptable carrier. Based on the routes of administration, the pharmaceutical composition of the present invention comprises preferably from 0.1% to 100% in weight of the total weight of the active ingredient.

The term “pharmaceutically acceptable carrier” used herein refers to a carrier(s), diluent(s) or excipient(s) that is acceptable, in the sense of being compatible with the other ingredients of the formulation and not deleterious to the subject to be administered with the pharmaceutical composition. Any carrier, diluent or excipient commonly known or used in the field may be used in the invention, depending to the requirements of the pharmaceutical formulation. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents.

The composition of the present invention can provide the effect of rapid, continued, or delayed release of the active ingredient after administration to the patient. According to the invention, the pharmaceutical composition may be adapted for administration by any appropriate route, including but not limited to oral, rectal, nasal, topical, vaginal, or parenteral route (such as intramuscular, intravenous, subcutaneous, and intraperitoneal), transdermal, suppository, and intranasal methods.

Regarding parenteral administration, it is preferably used in the form of a sterile water solution, which may comprise other substances, such as salts or glucose sufficient to make the solution isotonic to blood. The water solution may be appropriately buffered (preferably with a pH value of 3 to 9) as needed. Preparation of an appropriate parenteral composition under sterile conditions may be accomplished with standard pharmacological techniques well known to persons skilled in the art.

In one particular example of the invention, the pharmaceutical composition is formulated for oral administration. Such formulations may be prepared by any method known in the art of pharmacy. According to the present invention, the form of said composition may be tablets, pills, powder, lozenges, packets, troches, elixers, suspensions, lotions, solutions, syrups, soft and hard gelatin capsules, suppositories, sterilized injection fluid, and packaged powder.

In the invention, the method and composition/pharmaceutical composition are effective in treating a virus infection through an inhibition of a cysteine protease in a virus, particularly an RNA-dependent virus. Accordingly, the invention also provides a method and composition/pharmaceutical composition for treatment and/or prevention of a virus infection through inhibition of a cysteine protease in a virus, comprising using the compounds disclosed herein or pharmaceutically acceptable salt thereof.

Exemplified viruses which are responsive include, without limitation, a hepatitis virus, an influenza virus, a herpes simplex virus, an enterovirus, a rotavirus, a dengue virus, a poxvirus, a human immunodeficiency virusor, an adenovirus, a coronavirus infection, an arenavirus infection, a measles virus, a coronavirus or a norovirus. Preferably, the virus is a hepatitis virus, including a hepatitis B virus (HBV), a hepatitis C virus (HCV), a hepatitis D virus (HDV), or a SARS-CoV-2.

In another aspect, the present invention provides a method for treating or preventing an RNA-dependent virus infection through inhibiting a cysteine protease in a virus. One example of the virus is an RNA-dependent virus, such as SARS, MERS and SARS-COV-2; particularly SARS-COV-2.

In one further aspect, the present invention provides a composition/pharmaceutical composition for treating and/or preventing a virus infection through inhibiting a cysteine protease in a virus, which comprises any of the compounds disclosed herein, pharmaceutically acceptable salt thereof, or its mixture. Optionally, the composition/pharmaceutical composition may comprise at least one additional anti-viral therapeutic agent.

In one further aspect, the present invention provides a composition/pharmaceutical composition for treating and/or preventing a virus infection through inhibiting a sodium taurocholate cotransporting polypeptide (NTCP) in a cell, which comprises any of the compounds disclosed herein, pharmaceutically acceptable salt thereof, or its mixture. Optionally, the composition/pharmaceutical composition may comprise at least one additional anti-viral therapeutic agent.

In one further aspect, the present invention provides a use of any of the compounds disclosed herein for manufacturing a medicament for treating or preventing a virus infection through inhibiting a cysteine protease in a virus.

In one further aspect, the present invention provides a use of any of the compounds disclosed herein for manufacturing a medicament for treating or preventing a virus infection through inhibiting a sodium taurocholate cotransporting polypeptide (NTCP) in a cell.

In another aspect, the present invention provides a method for treating or preventing an DNA-dependent virus infection through inhibiting a sodium taurocholate cotransporting polypeptide (NTCP) in a cell. One example of the virus is an DNA-dependent virus, such as a Hepatitis B virus (HBV), Hepatitis C virus (HCV) and Hepatitis D virus (HDV).

The present invention is further illustrated by the following examples, which are provided for the purpose of demonstration rather than limitation.

### EXAMPLES

### Materials and Methods

I. FRET Protease Assays with the SARS-CoV-2 3CLpro

The establishment of an ED-FRET platform follows the protocol given by Jo et al. (2020). Briefly, a custom proteolytic, fluorogenic peptide with DABCYL and EDANS on ends, DABCYL-TSAVLQSGFRKMG-EDANS (Genomics, Taiwan), contains the consensus nsp4/nsp5 cleavage sequence that can be recognized by 3CLpro of SARS-CoV-2. The peptide is dissolved in distilled water and incubated with 3CLpro of SARS-CoV-2. Measurements of the spectral-based fluorescence are determined by a SPARK® multimode microplate reader provided by TECAN. The proteolytic activity is determined at 37° C. by fluorescent intensity of EDANS upon peptide hydrolysis as a function of time, in which λ_{excitation}=340 nm, λₑₘᵢₛₛᵢₒₙ=490 nm, bandwidths=9, 15 nm, respectively. Prior to the assay, the emission wavelength of the testing drugs at 340 nm excitation is examined to ensure that it does not overlap with the emission spectrum of EDANS.

Assays are conducted in triplicate in black 96-well microplates (Greiner) in 100 μL assay buffers (50 mM Tris pH 6.5) containing 0.25 μM SARS-CoV-2 3CLpro and 0.6 μM customized IQF substrate peptide.

II. Real-Time FRET Protease Assays with the SARS-CoV-2 3CLpro and Dose-Response Curve Analysis.

Prior to the addition of IQF peptide substrates, 0.25 μM SARS-CoV-2 3CLpro was incubated with a compound of interest at the indicated concentration (0-100 μM) in the assay buffer for an hour at 37° C. (SC-HM100, Sheng Ching Enterprise Co. Ltd). After that, 6 μM IQF peptide substrate was added to the mixture at the in a black 96-well microtiter plate immediately before the RFU detection in a TECAN SPARK® multimode microplate reader. Minor changes in the measurement parameters were made, as opposed to those used in protein activity assays. During the run, ten detection cycles at the gain value of 80 were performed, with an interval of 1.5 minutes. The change in fluorescence intensity was calculated by subtracting the initial value of a condition from its end value. Later, the change in fluorescence intensity per condition was normalized to the change in the negative control (vehicle only) in each assay plate. For each drug, the dose-response points at 0-100 μM were fitted to a normalized dose-response model given in GraphPad Prism 7.03 (GraphPad Software),

$$Y = Bottom + \frac{Top - Bottom}{1 + {10}^{( LogIC50 - X ) \cdot HillSlope}} .$$

III. Inhibition Assays in the Presence of Arjil Drugs.

The prospecting 13 Arjil drugs are pre-incubated with the SARS-CoV-2 3CLpro at 37° C. for 1 h. Those manifesting inhibitory activity against 3CLpro of SARS-CoV-2 will be investigated further at different concentrations to characterize their IC50 values, using GraphPad Prism 7.03 (GraphPad Software, San Diego, CA, USA). The prospecting 13 Arjil drugs are given in the table below.

| Compound No. | Compound Name and Structure |
|---|---|
| AR100-DS1 | 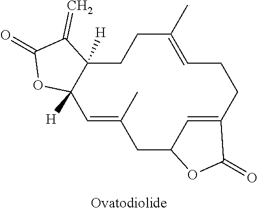 [SMILES: C=C1C(=O)O[C@H]2/C=C(\C)CC3C=C(CC/C=C(\C)CC[C@H]12)C(=O)O3] |
| AR101-DS1 | 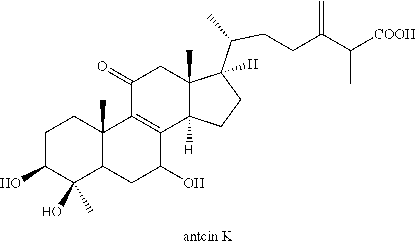 [SMILES: C=C(CC[C@@H](C)[C@H]1CC[C@H]2C3=C(C(=O)C[C@]12C)[C@@]1(C)CC[C@H](O)[C@](C)(O)C1CC3O)C(C)C(=O)O] |
| AR101-DS2 | 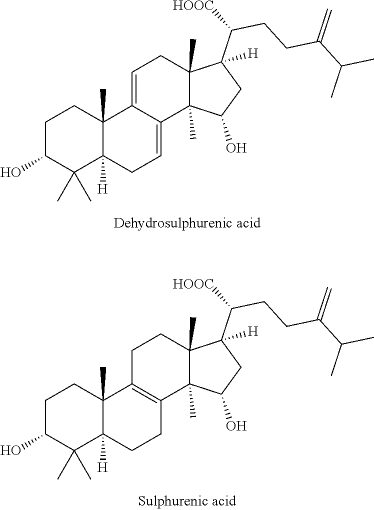 [SMILES: C=C(CC[C@@H](C(=O)O)[C@H]1C[C@H](O)[C@@]2(C)C3=CC[C@H]4C(C)(C)[C@H](O)CC[C@]4(C)C3=CC[C@]12C)C(C)C ; C=C(CC[C@@H](C(=O)O)[C@H]1C[C@H](O)[C@@]2(C)C3=C(CC[C@]12C)[C@@]1(C)CC[C@@H](O)C(C)(C)[C@@H]1CC3)C(C)C] |
| AR101-DS3 | 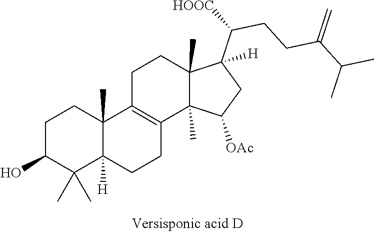 [SMILES: C=C(CC[C@@H](C(=O)O)[C@H]1C[C@H](OC(C)=O)[C@@]2(C)C3=C(CC[C@]12C)[C@@]1(C)CC[C@H](O)C(C)(C)[C@@H]1CC3)C(C)C] |
| AR101-DS4 | 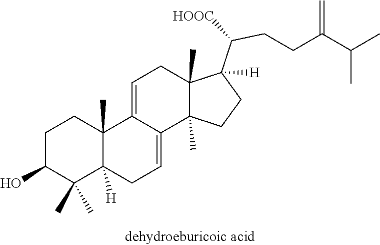 |
| ARH 020-DS1 | 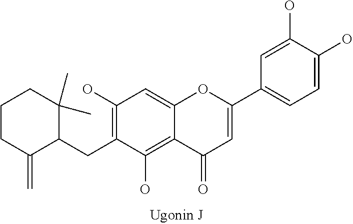 [SMILES: C=C1CCCC(C)(C)C1Cc1c(O)cc2oc(-c3ccc(O)c(O)c3)cc(=O)c2c1O] |
| ARH 020-DS2 | 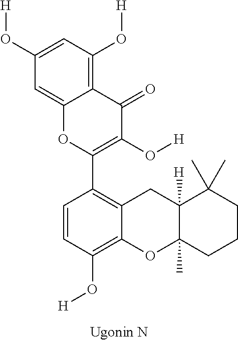 [SMILES: CC1(C)CCC[C@@]2(C)Oc3c(O)ccc(-c4oc5cc(O)cc(O)c5c(=O)c4O)c3C[C@@H]12] |
| ARH 019-DS1 | 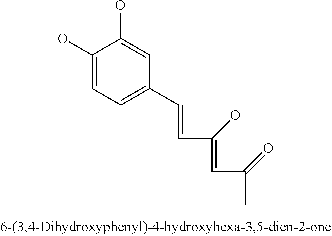 [SMILES: CC(=O)/C=C(O)/C=C/c1ccc(O)c(O)c1] |
| ARH 019-DS2 | 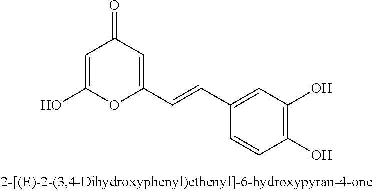 [SMILES: O=c1cc(O)oc(/C=C/c2ccc(O)c(O)c2)c1] |
| ARH 007-DS3 | 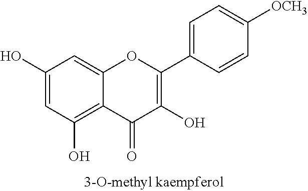 [SMILES: COc1ccc(-c2oc3cc(O)cc(O)c3c(=O)c2O)cc1] |
| ARH 007-DS4 | 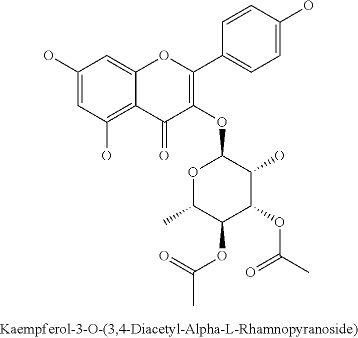 [SMILES: CC(=O)O[C@@H]1[C@@H](OC(C)=O)[C@@H](O)[C@H](Oc2c(-c3ccc(O)cc3)oc3cc(O)cc(O)c3c2=O)O[C@H]1C] |
| ARH 007-DS5 | 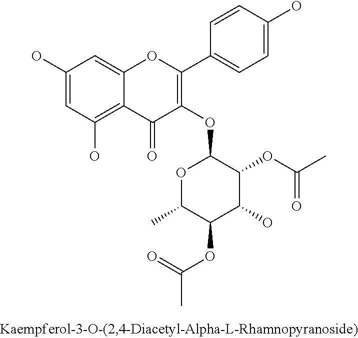 [SMILES: CC(=O)O[C@@H]1[C@@H](O)[C@@H](OC(C)=O)[C@H](Oc2c(-c3ccc(O)cc3)oc3cc(O)cc(O)c3c2=O)O[C@H]1C] |
| ARH013-DS1 | 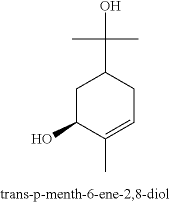 |

Prospecting Results

Based the knowledge and sequence-based SARS-CoV-2 3CLpro, the efficacy of 3CLpro inhibiting drugs provided by Arjil are assessed in vitro to determine their therapeutic potential in SARS-CoV-2 treatment. Concerning that no drug or vaccine has yet been approved to treat human SARS-CoV-2 infection, developing a broad-spectrum antiviral agent to combat against SARS-CoV-2 is of utmost importance and urgency. Enactment of ED-FRET technology and its workflow provides a robust, high-throughput drug discovery in the lab. Meanwhile, identification of SARS-CoV-2 3CLpro inhibiting agents from 13 tests proposed and provided by Arjil acts as guidelines of probable therapeutic doses in clinical assessment and prompts patent application in the future, contributing to antiviral library construction.

The 13 tests includes:
1. AR100-DS1+AR101-DS2;
2. AR100-DS1+AR101-DS3;
3. ARH 020-DS1 (Real-Time);
4. ARH 020-DS2 (Real-Time);
5. ARH 019-DS1 (Real-Time);
6. ARH 019-DS2 (Real-Time);
7. ARH 007-DS3 (Real-Time);
8. ARH 007-DS4 (Real-time);
9. ARH 007-DS5 (Real-Time);
10. AR101-DS2+ARH013-DS1;
11. ARH007-DS3+ARH013-DS1
12. AR100-DS1+ARH007-DS3; and
13. ARH 013-DS1 (Real-Time).

IV. Hep2.2.15 Cells.

Continuous HBV proliferation can be achieved in HepG2.2.15 cells (RRID:CVCL_L855) stably transfected with the HBV genome of the adw2 subtype. HepG2.2.15 cells are used because of the unlimited supply and constant quality and were maintained in Dulbecco's modified Eagle medium (DMEM; Invitrogen) supplemented with 10% heat-inactivated fetal bovine serum (FBS; Thenmo) plus 100 units of penicillin and 100×g of streptomycin per ml (both from Invitrogen).

V. HuS-E/2 Cells.

HuS-E2 cells that retains primary hepatocyte characteristics even after prolonged culture are utilized for HBV infection. For HBV infection, HuS-E/2 cells were differentiated with 2% DMSO for 7 days, and virus particles were collected to infect and replicate in HuS-E/2 cells as described in our previous study [38]. These cells are useful to assay infectivity of HBV strains, and screening of anti-HBV agents.

VI. Collection of HBV Particles.

The culture medium from drug-treated HepG2.2.15 cells is clarified by centrifugation at 1,000×g at 4° C. for 10 min, and then the supernatant is layered on top of a 20% sucrose cushion (20% sucrose, 20 mM HEPES, pH 7.4, 0.1% bovine serum albumin [BSA]) and centrifuged at 197,000×g for 3 h at 4° C. to pellet the 111V particles, which are then concentrated 100 fold to detect HBV DNA.

VII. DNA and RNA Isolation, Reverse Transcription and Real-Time PCR

Total DNA is extracted with a Genomic DNA isolation kit (Nexttec Biotechnologie, Germany). Total RNA is isolated from cultured cells using TRIzol® reagent (Invitrogen). Reverse transcription is performed with the RNA templates, AMV reverse transcriptase (Roche), and oligo-dT primer. The products are subjected to real-time PCR with primer sets of specific genes and SYBR Green PCR Master Mix (Bio-Rad). The primer sets used for HBV core, HBsAg, cccDNA and GAPDH are described [3]. The results are analyzed with the iCycler iQ real-time PCR detection system (Bio-Rad). Plasmid p1.3HBcl is prepared at 10-fold dilutions (2*104-2*109 copies/ml) to generate a standard curve in parallel PCR reactions.

VIII. Enzyme-Linked Immunosorbent Assay (ELISA)

The HBsAg ELISA Kit (General Biologicals Corp.) are used to detect hepatitis B surface antigen (HBsAg) with the protocol suggested.

IX. PreS1-NTCP Pull Down Assay

Expression and purification of recombinant fusion proteins and the GST pull-down assay were performed as described previously [40]. Briefly, expression of GST fusion proteins in E. coli BL21(DE3) was induced with 0.5 mM isopropyl-β-D-thiogalactopyranoside, then the bacterial cells were lysed by sonication at 4° C. in PBS containing 1% Triton X-100 (PBST) and separated into soluble and insoluble fractions by centrifugation at 13,800 g for 10 min at 4° C. To perform GST pull-down assays, the soluble fraction of bacterial lysates containing GST-fusion proteins was incubated for 3 hours at 4° C. with glutathione-Sepharose 4B beads (GE Healthcare Bio-Sciences), then the beads were washed 3 times with PBST before being incubated at 4° C. overnight with Huh7 cell lysate with overexpressed IA-NTCP prepared by lysis in PBST containing a protease inhibitor cocktail (1 mM PMSF, 10 μg/ml of aprotinin, 1 μg/ml of pepstatin A, 1 μg/ml of leupeptin). The beads were then washed with PBST, resuspended in sample buffer (12.5 mM Tris-HCl, pH 6.8, 2% SDS, 20% glycerol, 0.25% bromphenol blue, 5% b-mercaptoethanol), and subjected to SDS-polyacrylamide gel electrophoresis and examined by Western blot analysis.

X. Statistical Analysis

All values are expressed as mean t SE. Each value is the mean of at least three experiments in each drug in vitro experiments. Student's t-test is used for statistical comparison. * indicates that the values are significantly different from the control (*, p<0.05; **, P<0.01; ***, P<0.00l).

III. Results

1. Characterization of the Half Maximal Inhibitory Concentration of Inhibitors

The half maximal inhibitory concentration toward SARS-CoV-2 3CLpro was characterized by treating Arjil drugs at the indicated concentrations ranging from 0 μM to 100 μM. The IC750 values of Arjil drugs/tests against SARS-CoV-2 were demonstrated as Table 1. As shown in Figures below, the IC50 value of each Arjil drugs/tests were shown. Taken together, the inhibitory effect of combination of AR00-DS1+AR101-S2, AR101-DS2+AR013-DS1, AR100-DS1+ARH007-DS3 and AR100−DS1+AR101-DS3 on SARS-CoV-2 3CLpro highlights their therapeutic potentials against COVID-19. Also, ARH 020-DS2, ARH019-DS2, and ARH 007-DS3 are most promising compounds for inhibiting 3CLpro of SARS-CoV-2.

TABLE 1
Inhibition of SARS-CoV-2 3CLpro by Arjil drugs

| Arjil drugs | IC₅₀ (μM) | Origins |
|---|---|---|
| 1. AR100-DS1 + AR101-DS2 | 3.065 | Combination |
| 2. AR100-DS1 + AR101-DS3 | 2.934 | Combination |
| 3. ARH 020-DS1 (Real-Time) | 14.93 | Helminthostachys |
| | | zeylanica |
| 4. ARH 020-DS2 (Real-Time) | 6.329 | Helminthostachys |
| | | zeylanica |
| 5. ARH 019-DS1 (Real-Time) | 19.21 | Sanghuangporus |
| | | sanghuang |
| 6. ARH 019-DS2 (Real-Time) | 2.487 | Sanghuangporus |
| | | sanghuang |
| 7. ARH 007-DS3 (Real-Time) | 11.61 | Zingiber zerumbet |
| 8. ARH 007-DS4 (Real-Time) | 18.85 | Zingiber zerumbet |
| 9. ARH 007-DS5 (Real-Time) | 48.22 | Zingiber zerumbet |
| 10. AR101-DS2 + ARH013-DS1 | 2.409 | Combination |
| 11. ARH007-DS3 + ARH013-DS1 | 18.2 | Combination |
| 12. AR100-DS1 + ARH007-DS3 | 8.646 | Combination |
| 13. ARH013-DS1(Real-Time) | 32.89 | Alpinia katsumadai |
| | | Hayata |

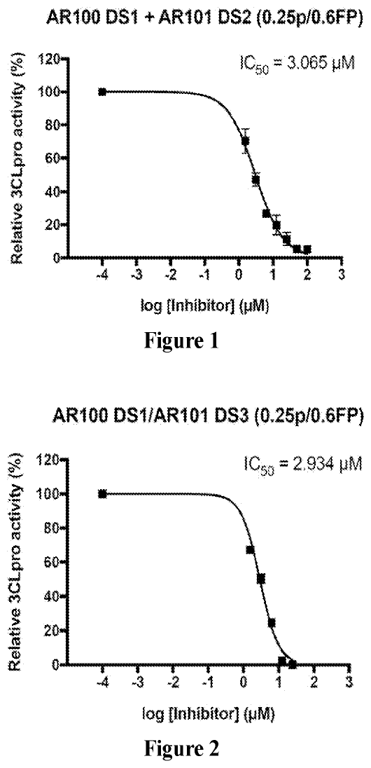

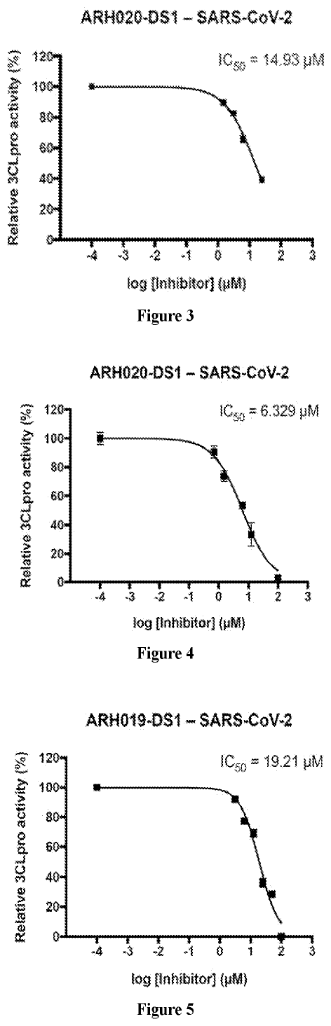

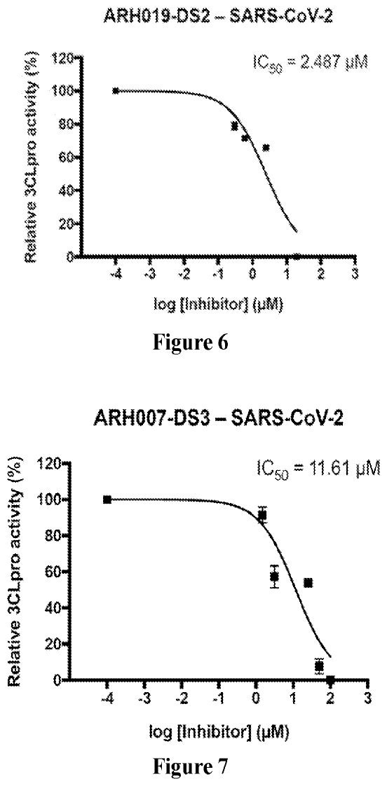

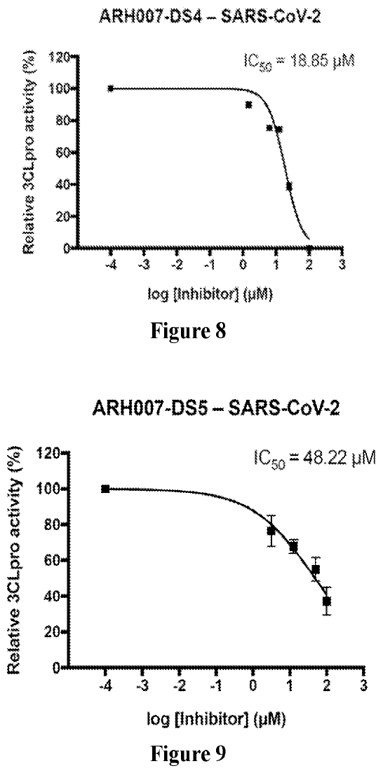

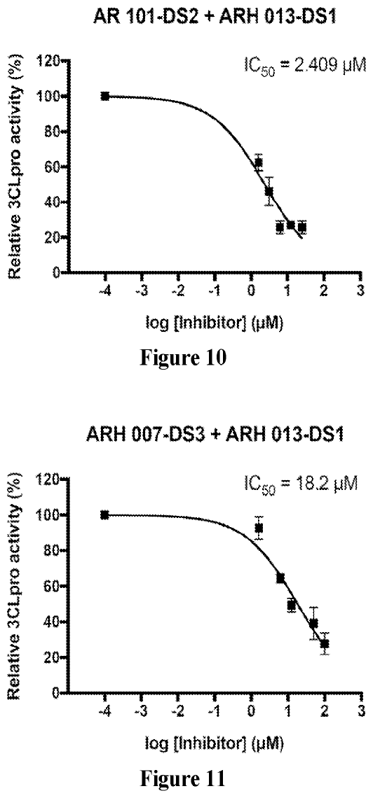

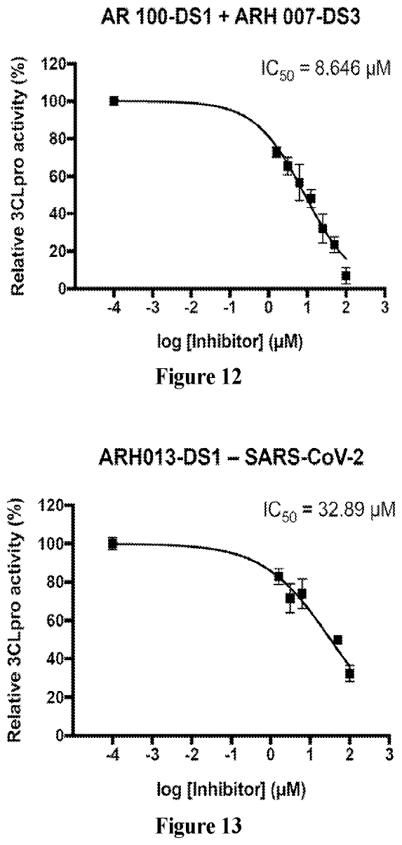

The IC50 values of ten example compounds/tests (AR1(0-DS1+AR101-DS2, AR100-DS1+AR101-DS3, ARH 020-DS1, ARH 020-DS2, ARH 019-DS1, ARH 019-DS2, ARH 007-DS3, ARt 007-DS4, ARH 007-DS5, ARH101-DS2+ARH013-DS1, ARH007-DS3+ARH013-DS1, AR100-DS1+ARH007-DS3, ARH013-DS1) were given in FIGS. 1-13.

As shown in FIG. 1, AR100-DS1+AR101-DS2 had an IC50 value of 3.065 μM in the presence of 0.25 μM SARS-CoV-2 3CLpro and 0.6 μM IQF peptide substrate (FP). Meanwhile, the inhibition of 0.25 μM AR100-DS1+AR101-DS3 on SARS-CoV-2 3CLpro and 0.6 μM IQF peptide substrate was determined (see FIG. 2), the IC50 value of AR100-DS1+AR101-DS3 against SARS-CoV-2 situated at 2.934 μM.

The IC50 values of ARH 020-DS2, ARH 019-DS2 and ARH 007-DS3 were 6.329 μM, 2.487 μM and 11.61 μM, respectively.

Given the above, the inhibitory effect of combination of AR100-DS1+AR101-DS2, AR101-DS2+ARH013-DS1, AR100-DS1+ARH007-DS3 and AR100-DS1+AR101-DS3 on SARS-CoV-2 3CLpro highlights their therapeutic potentials against COVID-19. Also, ARH 020-DS2, ARH 019-DS2, and ARH 007-DS3 are most promising compounds for inhibiting 3CLpro of SARS-CoV-2.

All publications, patents, and patent documents cited herein above are incorporated by reference herein, as though individually incorporated by reference.

The invention has been described with reference to various specific and preferred embodiments and techniques. However, one skilled in the art will understand that many variations and modifications may be made while remaining within the spirit and scope of the invention.

2. Effect of inhibitor on HBV secretion in HepG2.2.15 cells.

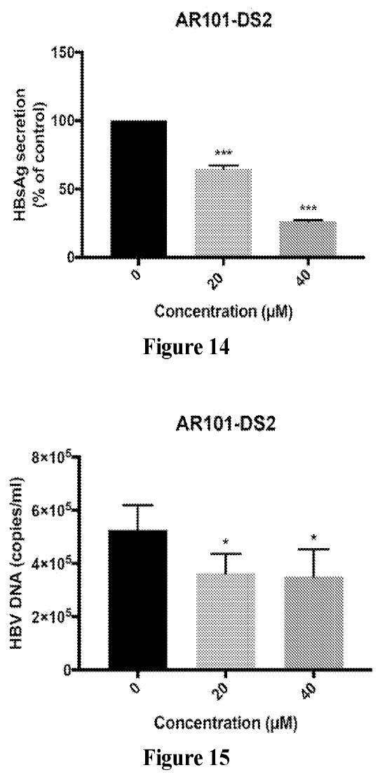

To test whether the above compounds had any effect on HBV genome replication, assembly, or secretion, HepG2.2.15 cells that were stably transfected with HBV genome, were used to incubate with AR101-DS2 for 48 hours, then HBsAg and 0.1-11V DNA collected from medium were measured by ELISA and real-time PCR. The results were shown in FIGS. 14 and 15.

The effects of AR101-DS2 on HBsAg secretion of HepG2.2.15 cells were shown in FIG. 14 (0, 20 and 40 μM of AR101-DS2). The secretion of HBsAg was significantly inhibited by the treatment of AR10l-DS2.

The effects of AR101-DS2 on the HBV DNA level in medium were shown in FIG. 15 (0, 20 and 40 μM of AR101-DS2). It was found that the DNA level was significantly reduced after the treatment of either 20 μM of AR101-DS2 or 40 μM of AR101-DS2. These results showed that AR101-DS2 suppressed HBV secretion in HepG2.2.15 cells

3. Effect of Inhibitor on HBV Infectivity of HuS-E/2 Cells.

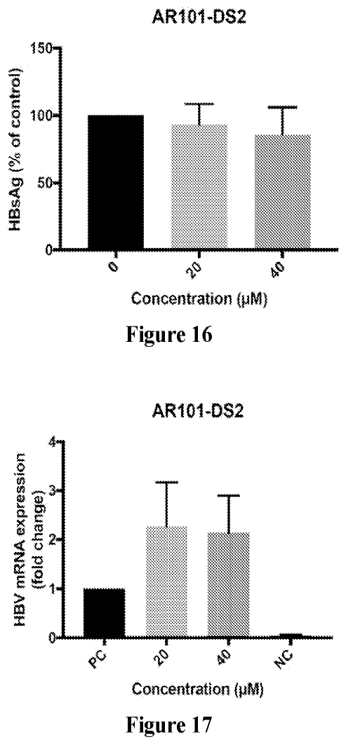

To evaluate the effects of AR101-DS2 on HBV infectivity and replication, HuS-E/2 cells were infected with any subtype HBV derived from HepG2.2.15 cells. The AR101-DS2 was added to the medium during infection with HBV for 18 h, then the infected cells were washed and incubated in fresh medium for 48 hours, when HBsAg in culture medium were detected by ELISA and HBV mRNA was detected by real-time PCR as an index of efficiency of HBV infection in HuS-E/2 cells. The results were shown in FIGS. 16 and 17.

The effects of AR101-DS2 on the entry of HBV in HuS-E/2 cells were shown in FIGS. 16 and 17. It was found that neither secretion of HBsAg in the medium nor HBV mRNA expression level showed dose-dependent reduction. Therefore, AR101-DS2 could not prevent HBV entering into HuS-E/2 cells.

4. The Inhibitor Effect on the Sodium Taurocholate Cotransporting Polypeptide (NTCP) in Cells.

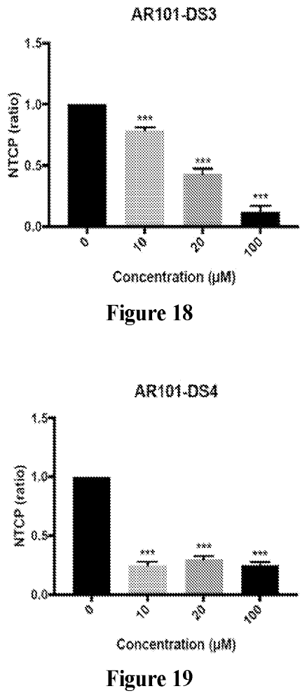

To evaluate the effects of inhibitor on inhibiting HBV infection, cell lysis extracted from cells treated with AR101-DS3 or AR101-DS4. The amount of NTCP shows a dose dependent increase from pull down assay as the amount of AR101-DS3 increase (0, 10, 20 and 100 g M, see FIG. 18).

Similarly, AR101-DS4 also caused a significant decreased in the amount of NTCP from pull down assay as showed in FIG. 19. These results showed that the inhibitors could inhibit hepatitis virus infection by inhibiting NTCP.

5. The Effect of Inhibitors Combination on HBV Infection Ability in HuS-E/2 Cells.

To evaluate whether multiple inhibitors combination would improve the ability on inhibiting HBV infectivity and replication, HuS-E/2 cells were infected with any subtype HBV derived from HepG2.2.15 cells. The AR101-DS1+AR101-DS3 or AR101-DS1+AR101-DS4 was added to the medium during infection with HBV for 18 h, then the infected cells were washed and incubated in fresh medium for 48 hours, when HBsAg in culture medium were detected by ELISA and HBV mRNA was detected by real-time PCR as an index of efficiency of HBV infection in HuS-E/2 cells.

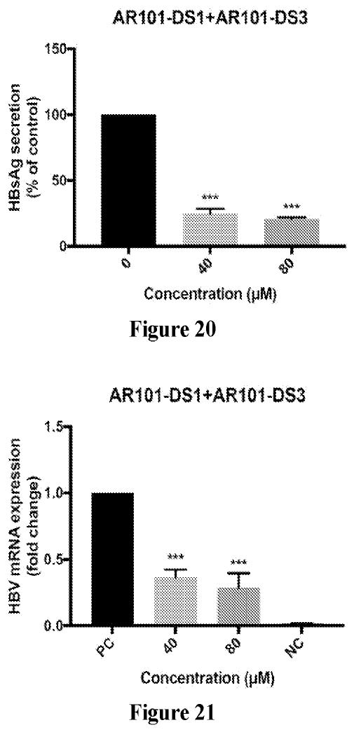

The effects of AR101-DS1+AR101-DS3 on the entry of HBV in HuS-E/2 cells were shown in FIGS. 20 and 21. It was found that both secretion of HBsAg in the medium and HBV mRNA expression level showed dose-dependent reduction. Therefore, AR101-DS1+AR101-DS3 could prevent HBV infection on HuS-E12 cells.

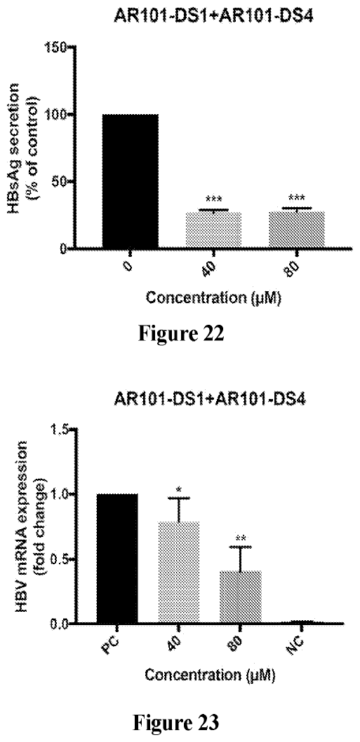

Similarly, the effects of AR101-DS1+AR101-DS4 on the entry of HBV in HuS-E/2 cells were shown in FIGS. 22 and 23. It was found that both secretion of HBsAg in the medium and HBV mRNA expression level showed dose-dependent reduction. Therefore, AR101-DS1+AR101-DS4 could prevent HBV infection on HuS-E12 cells.

While preferred embodiments of the present invention have been shown and described herein, it will be obvious to those skilled in the art that such embodiments are provided by way of example only and can be implemented in combinations. Numerous variations, changes, and substitutions will now occur to those skilled in the art without departing from the invention. It should be understood that various alternatives to the embodiments of the invention may be employed in practicing the disclosure. It is intended that the following claims define the scope of the invention and the methods and structures within the scope of these claims and their equivalents be covered thereby.

### REFERENCES

1. Ganem, D. and A. M. Prince, Hepatitis B virus infection-natural history and clinical consequences. N Engl J Med. 2004. 350(11): p. 1118-29.
2. Beasley, R. P., Hepatitis B virus. The major etiology of hepatocellular carcinoma. Cancer, 1988. 61(10): p. 1942-56.
3. Zoulím, F. and S. Locarníní, Hepatitis B virus resistance to nucleos(t)ide analogues. Gastroenterology, 2009. 137(5): p. 1593-608 e1-2.
4. Chen, W. N. and C. J. Oon, Human hepatitis B virus mutants: significance of molecular changes. FEBS Lett, 1999. 453(3): p. 237-42.
5. Yan, H., et al., Sodium taurocholate cotransporting polypeptide is a functional receptor for human hepatitis B and D virus. Elife. 1: p. e00049.
6. Watashi, K., et al., NTCP and beyond: opening the door to unveil hepatitis B virus entry. Int J Mol Sci. 15(2): p. 2892-905.
7. Gripon, P., et al., Infection of a human hepatoma cell line by hepatitis B virus. Proc Natl Acad Sci USA, 2002. 99(24): p. 15655-60.
8. Urban, S. and P. Gripon, Inhibition of duck hepatitis B virus infection by a myristoylated pre-S peptide of the large viral surface protein. J Virol, 2002. 76(4): p. 1986-90.
9. Abou-Jaoude, G., et al., Myristoylation signal transfer from the large to the middle or the small HBV envelope protein leads to a loss of HDV particles infectivity. Virology, 2007. 365(1): p. 204-9.
10. Chai, N., et al., Assembly of hepatitis B virus envelope proteins onto a lentivirus pseudotype that infects primary human hepatocytes. J Virol, 2007. 81(20): p. 10897-904.

11. Gudima, S., et al., Primary human hepatocytes are susceptible to infection by hepatitis delta virus assembled with envelope proteins of woodchuck hepatitis virus. J Virol, 2008. 82(15): p. 7276-83.
12. Corey L, Spear P G. Infections with herpes simplex viruses. New Engl J Med1986; 314: 686-691.
13. Ahmed R, Morrison L A, Knipe D M. Persistence of viruses. In: Fields B N, Knipe D M, Howley P M, eds. Field's Virology. Philadelphia: Lippincott-Raven Publishers, 1996: 219-250.
14. Contreras A, Slots J. Herpesviruses in human periodontal disease. J Periodontal Res2000; 35: 3-16.
15. Parra B, Slots J. Detection of human viruses in periodontal pockets using polymerase chain reaction. Oral Microbiol Immunol 1996; 11:289-293.
16. Ling I.-J, Ho C-C, Wu C-Y, Chen Y-T, Hung S-L. Association between human herpesviruses and the severity of periodontitis. J Periodontol 2004; 75: 1479-1485.
17. Contreras A, Slots J. Mammalian viruses in human periodontitis. Oral Microbiol Immunol 1996; 11: 381-386.
18. Park N H. Virology, In: Nisengard R J, Newman M G, eds. Oral Microbiology and Immunology. Philadelphia: W. B. Saunders Company, 1994: 248-285.
19. Yura Y, Iga H, Kondo Y, et al. Herpes simplex virus type 1 and type 2 infection in human oral mucosa in culture. J Oral Pathol Med 1991; 20: 68-73.
20. Rones Y, Hochman N, Ehrlich J, Zakay-Rones Z. Sensitivity of oral tissues to herpes simplex virus—in vitro. J Periodontol 1983; 54: 91-95.
21. Hung S-L, Wang Y-H, Chen H-W, Lee P-L, Chen Y-T. Analysis of herpes simplex virus entering into cells of oral origin. Virus Res2002; 86: 59-69.
22. Furman P A, St Clair M H, Spector T. Acyclovir triphosphate is a suicide inactivator of the herpes simplex virus DNA polymcrase. J Biol Chem 1984; 259: 9575-9579.
23. Morfin F, Thouvenot D. Herpes simplex virus resistance to antiviral drugs. J Clin Virol 2003; 26:29-37.
24. Field H J. Herpes simplex virus antiviral drug resistance-current trends and future prospects. J Clin Virol 2001; 21: 261-269.
25. Chen, C.-N., Lin, C. P. C., Huang, K.-K., Chen, W.-C., Hsieh, H.-P., Liang, P.-H., & Hsu, J. T. A. (2005). Inhibition of SARS-CoV 3C-like Protease Activity by Theaflavin-3,3′-digallate (TF3). Evidence-based complementary and alternative medicine: eCAM, 2(2), 209-215. doi:10.1093/ecam/neh081
26. Hegyi, A., Friebe, A., Gorbalenya, A. E., & Ziebuhr. J. (2002). Mutational analysis of the active centre of coronavirus 3C-like proteases. Journal of General Virology, 83(3), 581-593.
27. Needle, D., Lountos, G. T., & Waugh, D. S. (2015). Structures of the Middle East respiratory syndrome coronavirus 3C-like protease reveal insights into substrate specificity. Acta Crystallographica Section D: Biological Crystallography, 71(5), 1102-1111.
28. Herold, J., Gorbalenya, A. E., Thiel, V., Schelle, B., & Siddell, S. G. (1998). Proteolytic processing at the amino terminus of human coronavirus 229E gene 1-encoded polyproteins: identification of a papain-like proteinase and its substrate. Journal of Virology, 72(2), 910-918.
29. Jo, S., Kim, S., Shin, D. H., & Kim, M.-S. (2020). Inhibition of SARS-CoV 3C L protease by flavonoids. Journal of enzyme inhibition and medicinal chemistry, 35(1), 145-151.
30. Coronavirus disease 2019 (SARS-CoV-2) Situation Report-28. (2020). Retrieved from https://www.who.int/emergencies/diseases/novel-coronavirus-2019/situation-reports:
31. Li, G., & De Clercq, E. (2020). Therapeutic options for the 2019 novel coronavirus (SARS-CoV-2). In: Nature Publishing Group.
32. De Clercq, E., & Li, G. (2016). Approved antiviral drugs over the past 50 years. Clinical microbiology reviews, 29(3), 695-747.
33. Liu, W., Morse, J. S., Lalonde, T., & Xu, S. (2020). Learning from the Past: Possible Urgent Prevention and Treatment Options for Severe Acute Respiratory Infections Caused by 2019-nCoV. ChemBioChem.
34. Zumla, A., Chan, J. F., Azhar, E. I., Hui, D. S., & Yuen, K.-Y. (2016). Coronaviruses—drug discovery and therapeutic options. Nature reviews Drug discovery, 15(5), 327.
35. Wang, M., Cao, R., Zhang, L., Yang, X., Liu, J., Xu, M., . . . Xiao, G. (2020). Remdesivir and chloroquine effectively inhibit the recently emerged novel coronavirus (SARS-CoV-2) in vitro. Cell Research, 1-3.
36. Feng, J. Y. (2018). Addressing the selectivity and toxicity of antiviral nucleosides. Antiviral Chemistry and Chemotherapy, 26, 2040206618758524. doi:10.1177/2040206618758524
37. Jean, F., Basak, A., DiMaio, J., Seidah, N., & Lazure, C. (1995). An internally quenched fluorogenic substrate of prohormone convertase 1 and furin leads to a potent prohormone convertase inhibitor. Biochemical Journal, 307(3), 689-695.
38. Huang, H. C., et al., Entry of hepatitis B virus into immortalized human primary hepatocytes by clathrin-dependent endocytosis. J Virol. 86(17): p. 9443-53.
39. Lin S C, Liu C J, Chiu C P, Chang S M. Lu S Y, Chen Y J. Establishment of OC3 oral carcinoma cell line and identification of N F-kappa B activation responses to areca nut extract. J Oral Pathol Med2004; 33: 79-86.
40. Huang C, Chang S C, Yu I C, Tsay Y G, Chang M F. 2007. Large hepatitis delta antigen is a novel clathrin adaptor-like protein. Journal of Virology 81:5985-5994.

1. A method for inhibiting a virus infection comprising administering to a subject in need thereof a pharmaceutical composition comprising a therapeutically effective amount of a compound or pharmaceutically acceptable salt thereof, or its mixture, in which the compound is selected from the group consisting of:
Ugonin J having the structure of formula I and its derivatives: 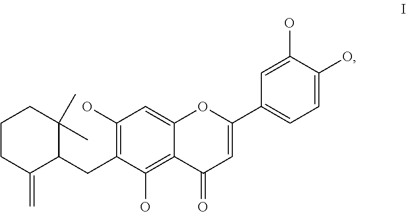
Ugonin N having the structure of having the structure of formula II and its derivatives: 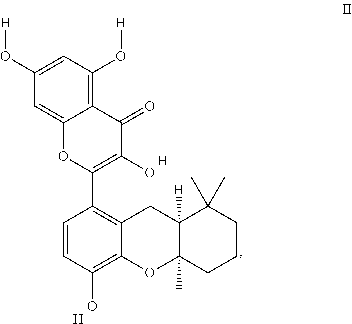
6-(3,4-dihydroxyphenyl)-4-hydroxyhexa-3,5-dien-2-one having the structure of formula III and its derivatives: 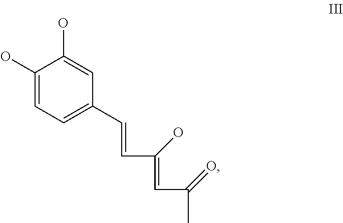 [SMILES: CC(=O)/C=C(O)/C=C/c1ccc(O)c(O)c1]
2-[(E)-2-(3,4-dihydroxyphenyl)ethenyl]-6-hydroxypyran-4-one having the structure of formula IV and its derivatives: 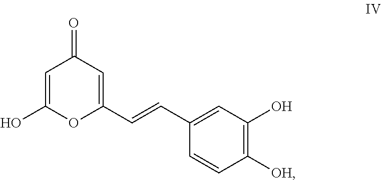 [SMILES: O=c1cc(O)oc(/C=C/c2ccc(O)c(O)c2)c1]
dehydroeburicoic acid having the structure of formula V and its derivatives: 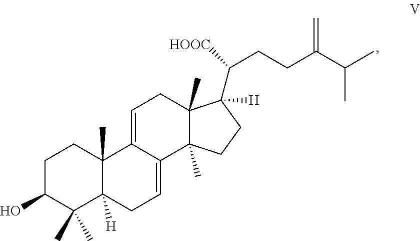
3-O-methyl kaempferol having the structure of formula VI and its derivatives: 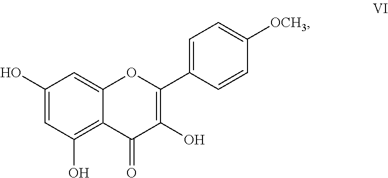 [SMILES: COc1ccc(-c2oc3cc(O)cc(O)c3c(=O)c2O)cc1]
kaempferol-3-O-(3, 4-diacetyl-Alpha-L-rhamnopyranoside having the structure of formula VII and its derivatives: 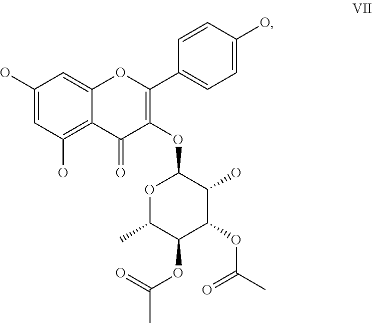
kaempferol-3-O-(2, 4-diacetyl-Alpha-L-rhamnopyranoside having the structure of formula VIII and its derivatives: 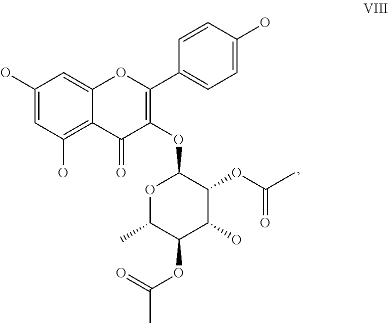 [SMILES: CC(=O)O[C@@H]1[C@@H](O)[C@@H](OC(C)=O)[C@H](Oc2c(-c3ccc(O)cc3)oc3cc(O)cc(O)c3c2=O)O[C@H]1C]
dehydrosulphurenic acid having the structure of formula IX and its derivatives: 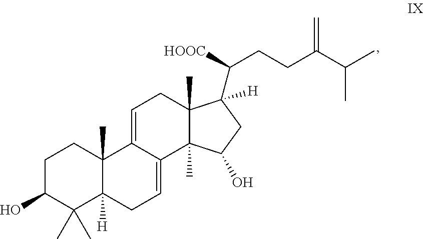 [SMILES: C=C(CC[C@H](C(=O)O)[C@H]1C[C@H](O)[C@@]2(C)C3=CC[C@H]4C(C)(C)[C@@H](O)CC[C@]4(C)C3=CC[C@]12C)C(C)C]
sulphurenic acid having the structure of formula X and its derivatives: 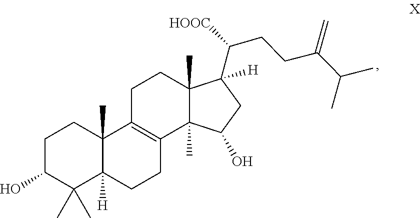 [SMILES: C=C(CC[C@@H](C(=O)O)[C@H]1C[C@H](O)[C@@]2(C)C3=C(CC[C@]12C)[C@@]1(C)CC[C@@H](O)C(C)(C)[C@@H]1CC3)C(C)C]
versisponic acid D having the structure of formula XI and its derivatives: 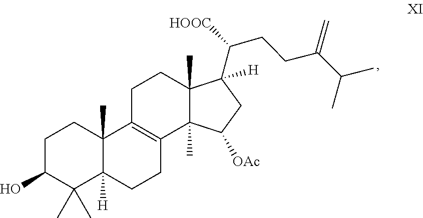 [SMILES: C=C(CC[C@@H](C(=O)O)[C@H]1C[C@H](OC(C)=O)[C@@]2(C)C3=C(CC[C@]12C)[C@@]1(C)CC[C@H](O)C(C)(C)[C@@H]1CC3)C(C)C]
trans-p-menth-6-ene-2,8-diol having the structure of formula XII and its derivatives: 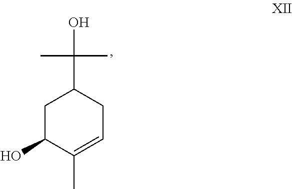
antcin K having the structure of formula XIII and its derivatives: 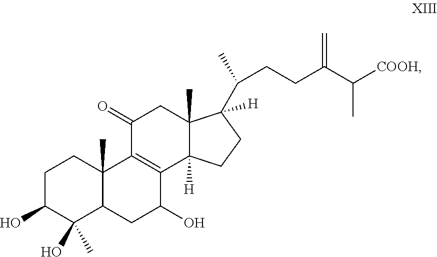 [SMILES: C=C(CC[C@@H](C)[C@H]1CC[C@H]2C3=C(C(=O)C[C@]12C)[C@@]1(C)CC[C@H](O)[C@](C)(O)C1CC3O)C(C)C(=O)O]
and combination thereof.

2. The method of claim 1, in which the subject is administered with a combination of two or more of the compounds.

3. The method of claim 1, in which the compound is selected from the group consisting of Ugonin J, Ugonin N, 6-(3,4-Dihydroxyphenyl)-4-hydroxyhexa-3,5-dien-2-one, 2-[(E)-2-(3,4-dihydroxyphenyl)ethenyl]-6-hydroxypyran-4-one, dehydroeburicoic acid, 3-O-methyl kaempferol, kaempferol-3-O-(3, 4-Diacetyl-Alpha-L-rhamnopyranoside, kaempferol-3-O-(2, 4-Diacetyl-Alpha-L-rhamnopyranoside, dehydrosulphurenic acid, sulphurenic acid, versisponic acid D and trans-p-Menth-6-ene-2,8-diol, and antcin K.

4. The method of claim 2, in which the compounds are selected from the group consisting of Ugonin J, Ugonin N, 6-(3,4-Dihydroxyphenyl)-4-hydroxyhexa-3,5-dien-2-one, 2-[(E)-2-(3,4-dihydroxyphenyl)ethenyl]-6-hydroxypyran-4-one, dehydroeburicoic acid, 3-O-methyl kaempferol, kaempferol-3-O-(3, 4-Diacetyl-Alpha-L-rhamnopyranoside, kaempferol-3-O-(2, 4-Diacetyl-Alpha-L-rhamnopyranoside, ovatodiolide, dehydrosulphurenic acid, sulphurenic acid, versisponic acid D and trans-p-Menth-6-ene-2,8-diol, and antcin K.

5. The method of claim 1, wherein the virus is a hepatitis B virus, a hepatitis C virus, or a hepatitis D virus.

6. The method of claim 5, wherein the virus is a hepatitis B virus (HBV).

7. The method of claim 1, wherein the virus is a herpes simplex virus (HSV).

8. The method of claim 1, wherein the virus is a coronavirus.

9. The method of claim 8, wherein the coronavirus is selected from the group consisting of severe acute respiratory syndrome (SARS), Middle East respiratory syndrome (MERS), and SARS-COV-2.

10. The method of claim 8, wherein the coronavirus is SARS-COV-2.

11. The method of claim 1, in which the compound is effective in inhibiting a cysteine protease in a virus.

12. The method of claim 1, in which the compound is effective in inhibiting a sodium taurocholate cotransporting polypeptide (NTCP) in a cell.

13. The method of claim 1, further comprising administering at least one additional anti-virus therapeutic agent.

14. A method for treating or preventing a virus infection through inhibiting a cysteine protease in a virus comprising administering to a subject in need thereof a composition/pharmaceutical composition comprising a compound or pharmaceutically acceptable salt, or its mixture, in which the compound is the compound as set forth in claim 1.

15. A method of claim 14, wherein the compound is effective in inhibiting a sodium taurocholate cotransporting polypeptide (NTCP) in a cell.

16. A combination/composition/pharmaceutical composition for treating or preventing a virus infection comprising a compound as set forth in claim 1, or pharmaceutically acceptable salt, or mixture thereof, at the amount effective to inhibit a cysteine protease in a virus, in combination of a pharmaceutically acceptable carrier.

17. A combination/composition/pharmaceutical composition for treating or preventing a virus infection comprising a compound as set forth in claim 1, or pharmaceutically acceptable salt, or mixture thereof, at the amount effective to inhibit sodium taurocholate cotransporting polypeptide (NTCP) in a cell.

18. The combination/composition/pharmaceutical composition of claim 16, wherein the virus is an RNA-dependent virus.

19. The combination/composition/pharmaceutical composition of claim 18, wherein the RNA-dependent virus is a coronavirus.

20. The combination/composition/pharmaceutical composition of claim 19, wherein the coronavirus is SARS, MERS or SARS-CoV-2.

21. The combination/composition/pharmaceutical composition of claim 19, wherein the coronavirus is SARS-CoV-2.

22. The combination/composition/pharmaceutical composition of claim 16, wherein the virus is a hepatitis B virus, a hepatitis C virus, or a hepatitis D virus.

23. The combination/composition/pharmaceutical composition of claim 22, wherein the virus is hepatitis B virus (HBV).

24. The combination/composition/pharmaceutical composition of claim 16, wherein the virus is a herpes simplex virus (HSV).

25-33. (canceled)
